(19) United States
(12) Patent Application Publication
(10) Pub. No.: US 2021/0048492 A1
(43) Pub. Date: Feb. 18, 2021

(54) FORWARD-LOOKING MRI COILS WITH METAL-BACKING

(71) Applicant: THE JOHNS HOPKINS UNIVERSITY, Baltimore, MD (US)

(72) Inventors: Ehud Schmidt, Towson, MD (US); Akila Viswanathan, Baltimore, MD (US); Henry R. Halperin, Baltimore, MD (US); Eric S. Meyer, Baltimore, MD (US); Akbar Akipour, Baltimore, MD (US); Seyed Hassan Elahi, Baltimore, MD (US); Wolfgang Loew, Cincinnati, OH (US)

(21) Appl. No.: 16/980,991

(22) PCT Filed: Mar. 15, 2019
(86) PCT No.: PCT/US2019/022460

§ 371 Date: Sep. 15, 2020

Related U.S. Application Data
(60) Provisional application No. 62/643,458, filed on Mar. 15, 2018.

(51) Int. Cl. G01R 33/34; A61B 5/00; A61B 5/055; G01R 33/36

(52) U.S. Cl. CPC G01R 33/34053; A61B 5/4337; A61B 5/4331; G01R 33/3685; A61B 5/004; G01R 33/34084; A61B 5/055

(57) ABSTRACT

An extended forward looking RF coil is shaped similarly to the tip of a pencil, for use in imaging and visualization of anatomy or certain conditions, including cancer. The RF coils are designed using the concept of image RF fields. The coils all include an inner void, which is surrounded by a cone-shaped plastic enclosure. A metallic layer is deposited on a surface of the cone and serves as a metallic layer backing the coils. The metallic layer includes a dielectric region, with altered size and geometry. Several solenoidal coil windings are placed outside the dielectric. The coil windings are denser on the Left (Forward) as compared to the Right (Backwards), in order to concentrate the magnetic field in the Forward direction. The extended forward looking coil is further integrated into an anatomy-specific, imaging array with sideways-looking RF coils, intended for improved imaging along cylindrical sides of the pencil-shaped device.

### CROSS REFERENCE TO RELATED APPLICATION

This application claims the benefit of U.S. Provisional Patent Application No. 62/643,458 filed on Mar. 15, 2018, which is incorporated by reference, herein, in its entirety.

### GOVERNMENT RIGHTS

This invention was made with government support under HL094610 awarded by the National Institutes of Health. The government has certain rights in the invention.

### FIELD OF THE INVENTION

The present invention relates generally to medical imaging machinery. More particularly, the present invention relates to forward-looking MRI coils with metal-backing.

### BACKGROUND OF THE INVENTION

Magnetic Resonance Imaging (MRI) employs radio-frequency (RF) coils in order to both excite (transmit energy) and thereafter receive signals from material, such as human tissue, that contains un-paired Nuclear Magnetic Resonance (NMR) spins. In some cases, a single coil is used for both transmission of signals to the spins and signal reception from the spins, but in most situations, separate coils are used for transmission and reception. The material which is imaged can be entirely physically enclosed by the coil, or it can be located outside of the region enclosed by the coil. In commercial MRI scanners which are intended for human imaging, a large diameter, RF coil, termed the body coil, which is designed to transmit a highly-homogeneous RF magnetic field, and surrounds the imaged anatomy, performs most excitation duties. However, organ-specific coils, which are optimized for imaging specific anatomical regions, are mostly used for reception, since they provide far larger sensitivity within a specified smaller and restricted region, permitting the acquisition of equivalent spatial-resolution MR images in shorter scan times. The spatial sensitivity of MRI RF coils is quantified in terms of their Signal-to-Noise Ratio (SNR). Most MRI receiver coils do not entirely envelope the tissue they image, so they are outward-looking coils. The sensitive (high SNR) restricted region that most MRI receiver coils see, if they are configured as circularly-shaped or rectangularly-shaped loop coils, extends away from them for a distance approximately the size of their diameter, or length, respectively. For example, most RF receiver coils intended for abdominal imaging are made of 5 or 6 cm diameter loops, and they then individually see approximately this distance into the human body. Since the cross-section of the abdomen is approximately 30 cm (Anterior-Posterior) by 40 cm (Left-Right) by 40 cm (Superior-Inferior), multiple surface coils are placed on the surface, in a matter that allows their sensitive regions to overlap at greater depths. They then individually deliver high signal in the surface regions of the abdomen, which are located physically closer to them, while the added contribution of multiple coils corrects for their lower individual sensitivity at greater distances.

Therefore, it would be advantageous to provide a forward-looking MRI coil that is sensitive at greater distances from its forward (or distal) tip, relative to conventional coils, as exemplified by a coil with metal-backing. Metal-backing, otherwise referred to as passive-shielding, is a method to change the sensitive region (or lobe pattern) of a coil. Metal-backing, by virtue of the fact that the net time-varying magnetic fields on a metallic surface must by zero, can create opposing fields to those induced on the surface, can lead to reduction in the net field in a direction that passes through the surface, while increasing the net field in a direction away from the surface.

In a practical organ-specific application, it may be required to merge one or more forward-looking coils with one or more sideways-looking coils, thus forming a coil array, which together deliver the desired spatial coverage.

### SUMMARY OF THE INVENTION

The foregoing needs are met, to a great extent, by the present invention, wherein in one aspect a device for magnetic resonance imaging includes an extended, forward-looking RF coil for use in imaging and visualization. The device includes a base for the extended, forward-looking RF coil. Further, the device includes a metallic layer deposited on the base, such that it provides a metallic backing for the extended, forward-looking RF coil.

In accordance with an aspect of the present invention, the device further includes the extended, forward looking RF coil including an inner void. The extended, forward looking RF coil is surrounded by the base taking a form of a cone-shaped plastic enclosure. The metallic layer is deposited on an outer surface of the cone. The device can take the form of an obturator used in the field of Radiation Oncology. Additionally, the device can include coil windings of the extended, forward-looking RF coil being denser on a left side (forward side) as compared to the right side (backwards side), which is required in order to focus the illumination of the coil in one direction. Capacitors are disposed in the coil windings. The device includes a matching, tuning, and decoupling circuit. The device can also include a braided metallic catheter including floating resonant radio-frequency traps (Baluns).

In accordance with another aspect of the present invention, a device for magnetic resonance imaging includes a forward-looking magnetic resonance imaging array, wherein the forward-looking magnetic resonance imaging array includes a metallic layer and coil windings disposed outside of the metallic layer. The device includes a sideways-looking magnetic resonance imaging array, wherein the sideways-looking magnetic resonance imaging array includes metallic coils disposed about a base. The coil windings of the forward-looking magnetic resonance imaging array are more dense at a distal end of the array that at a proximal end of the array.

In accordance with still another aspect of the present invention, there are capacitors disposed within the coil windings of the forward-looking magnetic resonance imaging array. The device includes an insulator layer disposed between the metallic layer and the coil windings of the forward-looking magnetic resonance imaging array. The forward-looking magnetic resonance imaging array takes a generally conical form. The coil windings of the sideways-looking array are disposed around a plastic cylinder. The coil windings of the sideways-looking array includes capacitors disposed among the coil windings. The sideways-looking magnetic resonance imaging array includes a four-element coil array. Each element of the four-element coil array covers a 90 degree arc. The device includes a matching, tuning, and decoupling circuit. Additionally, the device can include a braided metallic catheter including floating resonant radio-frequency traps (Baluns). The forward-looking magnetic resonance imaging array and the sideways-looking magnetic resonance imaging array are configured for imaging the vagina and cervix.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates an image view of a demonstration of High Dose Rate (HDR) Brachytherapy procedure for treatment of cervical cancer, showing insertion of catheters with radioactive material into a tumor. Dots indicate locations of radiation dose delivery. FIG. 1B illustrates a perspective view of devices used for HDR brachytherapy that includes a template and obturator.

FIGS. 2A and 2B illustrate side and perspective views of a design and components of a metal-backed extended forward-looking MRI coil.

FIGS. 3A and 3B illustrate a comparison of the radio-frequency magnetic field in a conventional, non-metallic backed coil with a metallic-backed coil.

FIGS. 4A and 4B illustrate image views of radio-frequency magnetic field simulated models for two different metal-backed geometries.

FIGS. 5A and 5B illustrate image views of radio-frequency magnetic field simulated models for metal-backed versus non-metal backed geometries for the optimally designed coil, which has a 5 mm radius opening on the Forward side, and the thickness of the dielectric layer varies from 5 mm on the Forward side to 2.5 mm on the Backward side.

FIG. 6A illustrates a side view and FIG. 6B illustrates a front view of a 1.5 Tesla forward-looking coil prototype without the external water-proofing cover layer, which includes some of the radio-frequency components.

FIG. 7A illustrates side views of the spatial coverage requirements of an active endovaginal obturator which contains an array of coils, including both forward-looking and sideways-looking coils. It shows a cartoon of such an array on the background of an MRI image of adult female genitourinary anatomy. FIG. 7B shows the design of the coil elements of this array and FIGS. 7C-7E show practical examples of the actual coverage of the array, as measured in sexually mature swine.

FIGS. 8A and 8B illustrate side views of the design and components of a pulled-back metal-backed focused forward-looking MRI coil.

FIGS. 9A and 9B illustrate image views of radio-frequency magnetic field simulated models for the metal-backed and pulled-back metal-backed MRI coils.

FIG. 10A illustrates a partially sectional view of a forward looking coil design, according to an embodiment of the present invention.

FIG. 10B illustrates an image view of the surface current distribution on the windings and on the metal surface for a forward looking coil design, according to an embodiment of the present invention.

FIG. 11 illustrates a side view of the RF active obturator array that includes both the forward looking coil and the sideways looking array, according to an embodiment of the present invention.

FIG. 12 illustrates a schematic diagram of four elements of a sideways-looking coil array, according to an embodiment of the present invention.

FIGS. 13A-13C illustrate side views of a prototype of a complete endo-vaginal array, including its shielded cabling for connection to the MRI scanner as well as mounted floating resonant radio-frequency traps, according to an embodiment of the present invention.

FIGS. 14A and 14B illustrate image views of phantom experiments with a prototype of the array along the transverse (plane perpendicular to the shaft orientation) and coronal (plane lying parallel to the shaft orientation) directions according to an embodiment of the present invention.

FIG. 15A illustrates a graphical view of measured SNR values along the forward direction along several spatial directions relative to the shaft orientation, according to an embodiment of the present invention.

FIG. 15B illustrates a graphical view of heating test results in an ASTM-standard gel phantom during 15 minutes of continuous 4 Watt/kg specific absorption rate MRI imaging, according to an embodiment of the present invention, and FIG. 15C illustrates the endovaginal active obturation array that yielded the results of FIG. 15B.

FIGS. 16A-16B illustrate image views from a swine model using the endovaginal array, placed in the swine vagina, highlighting the lobe patterns of the forward-looking and sideways looking elements, according to an embodiment of the present invention.

FIGS. 17A-17D illustrate image and graphical views of a comparison of the simulated magnetic field for a non-metallic backed coil, FIG. 17A with a metallic-backed coil, FIG. 17B. In FIG. 17C the solid line and dashed line show the magnetic field profiles of the metallic-backed coil and non-metallic backed coil, respectively, in the forward direction, which is along the shaft of the array, as defined in FIG. 17A-B. FIG. 17D illustrates the solid line and dashed line that show the magnetic field profiles of the metallic-backed coil and nonmetallic backed coil, respectively, along the oblique direction, as defined in FIG. 17A-B.

FIG. 18A illustrates a 2D Fast Spin Echo image of the Forward-looking coil in a CuSO₄-doped water solution phantom, acquired by combining the Forward-looking coil and the commercial MRI scanner's spine-array coils. FIG. 18B illustrates the calculated SNR profile in the forward direction of the forward-looking coil, which shows a 4-8 times surface-coil SNR enhancement.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides an extended forward looking RF coil for use in imaging and visualization of anatomy or certain conditions, including cancer. The extended forward-looking coils are designed using the concept of image RF magnetic fields. The coils define an inner void, which is filled with a cone-shaped plastic enclosure. A metallic layer is deposited on a surface of the cone, such that it is disposed on an inner surface of the cone. The metallic layer includes a dielectric region, whose size and geometry were altered, in order to optimize the desired performance, and outside the dielectric several solenoidal coil windings were placed. The coil windings are denser on the Left (Forward) side as compared to the Right (Backwards) side in order to concentrate the magnetic field in the Forward direction.

A special case of outward-looking coils occurs when the surface available for placing the coil is strongly limited, or restricted, due to practical utilization factors, but there is still a desire to image objects at a relatively large distance, extending over multiple coil diameters, away from the coil. Some examples of such constraints result from the shape of human anatomy. One example of such of a geometrical restriction is placement of a coil on the distal tip of a catheter, which needs to be smaller than the blood vessels through which it traverses (typically <3 mm), with the intended aim that the coil illuminates the region in front of the catheter. This presents a different problem than illuminating tissue along the sides of the catheter, since the catheter is very long (>1000 mm), which allows building simpler geometry coils on its sides for sideways-focused imaging. Therefore, these outward-looking coils will be herein categorized into forward-looking and sideways-looking coils. Another example is placement of an RF coil into the rectum, vagina or esophagus. These body orifices are narrow (<20 mm) and long (>200 mm). If it is desired to see the sides of these anatomies, sideways-looking coils are required, while if it is desired to see in front of the coils, forward-looking coils are required. Specifically, if a coil is placed into the vagina, with a diameter of ˜20 mm, its tip can be advanced within the vagina until it rests just below the cervix. As a result, a forward-looking coil for imaging the cervix gland, which is 20-30 mm from the coil's end, is relatively simple to construct, but if the goal is to image further above (e.g. 40-50 mm) the cervix, such as into the endometrium, that is a more difficult problem for current conventional-coil geometries. Coils intended for such applications will be referred to herein as extended forward-looking coils.

As a further example of an application of the present invention, an embodiment of the present invention is directed to interventional challenges created by advancing the catheters previously discussed into blood vessels, where there is injury to a section of the blood vessel, or the blood vessel is partially or totally occluded (such as in the Chronic Total Occlusion vascular application). While navigating through blood vessels, it is desirable to know well before (such as 20-40 mm) reaching the area of the total occlusion that it is close, because it may be necessary to proceed differently in its proximity, such as reducing the catheter advancement speed (so as not to perforate the vessel). In this case, an extended forward-looking coil is also preferable. Such extended forward-looking coils are alternatively referred to as flash-light RF coils.

In order to design effective extended forward-looking coils, the present invention leverages the concept of image RF fields. Image fields can be demonstrated by placing a circular loop of wire at a certain distance above the surface of a metal plate, oriented such that it is parallel to the plane of the surface. When there is an RF current running through the wire loop in the clockwise direction, a primary magnetic field is created. If one looks along a line that is perpendicular to the plane of the loop, and is centered at the center of the loop, it will be found that at locations between the loop and the metallic plate, the magnetic field principally points towards the metal plate (i.e. downwards), while at locations above the loop, the field principally points towards above the loop (i.e. upwards). These magnetic fields induce a transient magnetic field in the metal. The metal then builds a surface electrical current, which induces an opposing-directionality magnetic field, called an image magnetic field, which exactly cancels the primary field on the metal surface. As a result, there are now two fields, the primary magnetic field and an image magnetic field. In the region between the physical location of the loop and the metal, these two fields are in opposing directions and create a smaller net field, while above the loop, they are oriented in the same direction and therefore reinforce the primary field, creating a larger net field. This therefore contributes to a larger magnetic field at locations above the coil, relative to the case in which the metal plate were not present, leading to an extended field. The magnitude of the reinforcement and its spatial extent is controlled by several parameters, such as the distance between the loop and the metal, the material parameters (dielectric constant, electrical conductivity, magnetic permeability) of the region between the loop and the metal, as well as the properties of the metal and the shape of the metal surface. An additional important measure is the coil efficiency, otherwise referred to as the coil Quality factor (Q) which is the ratio of the RF current driven through the coil that is converted into electromagnetic energy, relative to the dissipated energy, since the transient electrical currents running on the metal surface result in the conversion of energy into heat through the coil's RF resistance. Bringing the wire loop very close to the metal would result in the largest forward extension of the RF field, but unfortunately also in a large increase in energy dissipation in the form of heat, due to very strong currents running on the metallic surface, so this design would form a very inefficient coil.

The primary focus of this invention is on designing and building an extended forward-looking RF coil which is placed in long, narrow orifices. More particularly, one exemplary implementation that is used herein as an illustration of the use of the present invention is as an extended forward-looking RF coil placed in the vagina and used for imaging the extent of tumor found in advanced cervical-cancer patients. In advanced cervical cancer, the cancer has spread from its primary location in the cervix, and is found also in the vagina and endometrium, so an extended visualization region is required in order to visualize and then treat the entire tumor.

Advanced cervical cancer consists of relatively large tumors that spread from the cervix into the endometrium and vaginal wall. It is treated in ˜40% of cases with radiation therapy, consisting of external beam radiation (EBRT) followed by high dose rate (HDR) interstitial radiation (brachytherapy). MR imaging is performed before brachytherapy to locate remnant tumors that survived EBRT. The goal is to deliver large focused radiation only to living tumor, and minimize radiation to surrounding tissues, which can cause severe side effects. Localizing surviving tumors post-EBRT is difficult, due to post-radiation reduced vascularity, hemorrhage and fibrosis. As a result, extensive MR imaging (T2, DWI, DCE, BOLD) is performed, to improve localization of the remnant tumor(s). This leads to long imaging times, since these tissues are positioned midway between the anterior body-array and the posterior spine-array, which reduces surface-coil Signal-to-Noise ratio (SNR). Placing a coil in the vaginal canal is attractive, since during brachytherapy, an obturator is inserted into the vagina to direct the trajectory of interstitial-catheters that are inserted into the tumors for radiation delivery.

Existing endo-vaginal MRI coils are diagnostic coils intended for imaging the vaginal wall or cervix, and do not meet the above requirements, primarily because their lobe patterns don't project upwards (in the Superior-Inferior direction) and therefore cannot illuminate the posterior-endometrium. “Flashlight” (forward-looking) lobe patterns that provide strong SNR at distances of 30-40 mm are difficult to deliver within the constraints of the <25 mm diameter vaginal-canal.

The present invention takes the form of a new imaging array, which includes elements for both sideways-looking (vaginal-wall) and forward-looking (cervix/posterior-endometrium) imaging. The coil is designed to be an “active obturator”, fulfilling the dual roles of supporting HDR-brachytherapy intervention and providing >4 times the SNR of the surface arrays. The “pencil” shaped endo-vaginal array has a cone at its top, for the forward-looking coil, and a cylindrical shaft, for the sideways-looking array. Its inner open lumen supports its obturator role. The forward-looking coil was designed utilizing the image-magnetic-field concept, wherein properly-positioned metallic surfaces force magnetic fields to project along selected directions. Design specifics were simulated and tested, since closely-placed metals can reduce the coil quality-factor (Q), which is the ratio of the stored (magnetic field) energy to the dissipated energy (in the form of heat). Finite-element electromagnetic simulations of the forward-looking coil (CST, Germany) evaluated the effect of the metallic surface on the magnetic-field surrounding the coil. The metallic surface shape, the distance between the metal and the solenoidal coil windings, and the winding diameter and spacing were all simulated. Optimal designs were then constructed and tested.

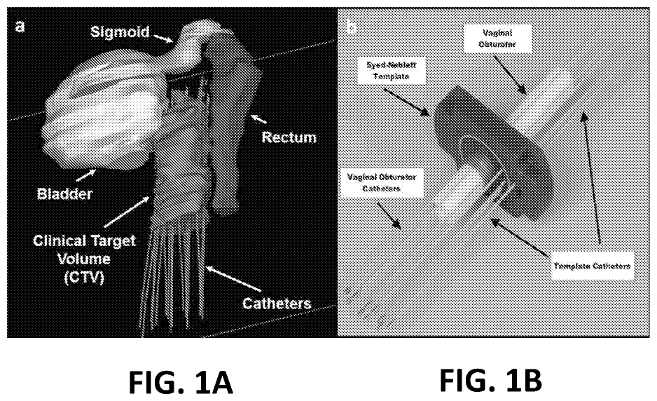

The coil of the present invention is intended to be used instead of a conventional vaginal obturator, which is used during High Dose Rate (HDR) radiation oncology brachytherapy procedures for tumor treatment, as illustrated in FIG. 1A. The vaginal obturator is a 20 mm outer diameter (OD) plastic cylinder which is inserted into the vagina, as illustrated in FIG. 1B. The obturator has holes in its center and along its surface for inserting brachytherapy catheters, which are thin (1.5-2.0 mm diameter) empty cylinders through which sources of radiation are later inserted. In an instance where the device takes the form of an active obturator, the device of the present invention can include the internal hole and the multiple external holes (for the metallic catheters that pass though the obturator). The present invention harnesses passive magnetic shielding instead of active RF shielding, primarily because actively shielded coils, which use coils to generate both the primary and the shielding magnetic fields, therefore require more space within the device.

FIG. 1A illustrates an image view of a demonstration of High Dose Rate (HDR) Brachytherapy for treatment of cervical cancer, showing insertion of catheters with radioactive material into a tumor. Dots indicate locations of radiation dose delivery. FIG. 1B illustrates a perspective view of a device used for HDR brachytherapy that includes a template. The template is attached to the exterior of the vaginal region and provides guiding holes through which the catheters are advanced. The brachytherapy catheters are advanced up to regions of the endometrium. Some of the brachytherapy catheters are also advanced through the sides of the obturator, which is a device which is inserted into the vagina in order to stabilize the location of the catheters. The intent is to replace the standard obturator with an active obturator, which can also serve as an RF receiver coil in order to image the region at higher sensitivity, in order to better detect remnant cancer. In advanced cervical cancer, because there is cancer on the sides of the vagina, in the cervix, as well as above the cervix, the imaging probe will need to visualize all these regions.

The intent of the present invention is to produce a coil array that has 4-6 times the SNR of commercial (abdominal and spine) surface RF coils currently employed in these procedures. The added SNR provided by the probe allows for high-resolution imaging in shorter scan times, which is required for detecting small remnant tumors that have survived the first application of radiation, which is commonly delivered using external beam radiation therapy (EBRT), and must be eradicated with the brachytherapy procedure, where focused higher-dose radiation is provided.

Note that similar designed coils, which are placed into body orifices or blood vessels, can be used in conjunction with commercial surface coils, in order to improve visualization of large regions of the pelvic, the abdomen, the gastro-intestinal system, the cardio-vascular system, or the lungs.

Similar devices may be used for:
1. MR Imaging of plaque within the lumen of blood vessels including CTO (Chronic Total occlusions), to prevent vessel puncture and safely open partially or totally occluded vessels during vascular interventional procedures.
2. MR Imaging of cardiac myocardial biopsies using active MRI-guided bioptomes (a catheter with jaws that grabs samples of cardiac tissues, so they can be removed for RFhistology) in order to increase biopsy yield.
3. MRI guided cryogenic, radio-frequency (inductive), laser, or focused ultrasound (FUS) ablation of the heart, with improved monitoring during the performance of therapy.
4. Performing MRI-guided trans-perineal cryogenic ablation (cryoablation), radio-frequency ablation (RFA) or focused ultrasound (FUS) ablation of the prostate, with improved visualization of the gland during the ablation procedure.

Additional advantages of coils with a metal backing;
1. Ability to insert the cables that lead the signal out of the coil to the MRI receiver into the region within the metal, which is shielded from external RF fields, so that the signal received by the coil from the material's spins is not corrupted by induced fields from the body coil. Similarly, other electronics that are sensitive to large RF fields (such as preamplifiers) can be placed inside this shielded region.

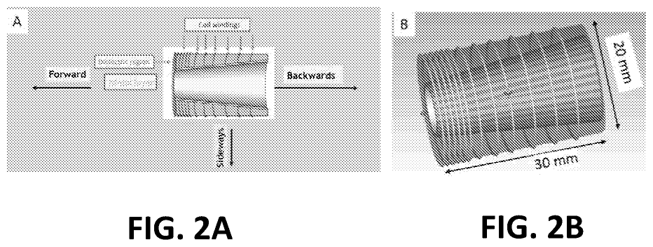

The magnetic fields created by RF coils of various designs, were first simulated, using electromagnetic simulation software packages (Computer Simulation Technology Inc., Germany). All the coils were designed with an emphasis on creating a magnetic field in front of the coil, as illustrated in FIG. 2A. All coils were constrained to 20 mm in outer diameter. The coils all consist of an inner void, which is surrounded by a cone-shaped plastic enclosure. On the outer surface of the cone, a metallic layer was created by placing a copper layer with a width of 30 micrometers, which is greater than 3 skin depths at the MRI Larmor frequencies (63.6/63.8 MHz for 1.5 Tesla scanners or 123.2/127.0 MHz for 3.0 Tesla scanners), and thereby establishes an effective metallic surface. On the outside of the metallic layer lies a dielectric (insulator) region, whose size and geometry were altered, and outside the dielectric several solenoidal coil windings were placed. The coil windings were denser on the Left (Forward) side as compared to the Right (Backwards) side, so as to concentrate the magnetic field in the Forward direction. The locations of the 12 coil windings, starting from the Forward side of the coil, are at: 0, 1, 2, 3, 4, 6, 9, 12, 15, 19, 23 mm. In order to compare the magnetic fields produced from coils with the metallic backing versus those without, the thin metal layer was replaced by an equivalent thickness dielectric layer, and the simulation repeated.

FIGS. 2A and 2B illustrate side and perspective views of a design and components of the metal-backed forward-looking MRI coil. FIG. 2A illustrates that the center of the coil is a void, surrounded by a thin-walled plastic cone. The cone is plated with a thin (30 micrometer) metal (copper) layer. Outside the metal is a dielectric region, on top of which a conductive wire is wound. Note that several parameters can be varied: (a) the angle of the cone (i.e. the internal diameter on the forward side), (b) the thickness of the dielectric layer from the Backwards to the Forward sides, and (c) the number and spatial distribution of the winds. The outer diameter of the complete device is 20 mm, and the length of the cone is 30 mm, and this is maintained in all designs. FIG. 2B illustrates a 3D view of the exterior of the coil. The locations of the 12 coil windings, starting from the forward side of the coil, is: 0, 1, 2, 3, 4, 6, 9, 12, 15, 19, 23 mm.

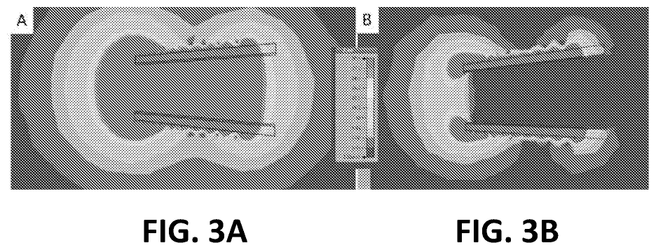

FIGS. 3A and 3B illustrate a comparison of the magnetic field in a conventional, non-metallic backed coil with a metallic-backed coil. FIG. 3A illustrates the non-metallic backed coil and FIG. 3B illustrates the metallic backed coil. Note that the magnetic field penetrates through the entire non-metallic-backed coil, whereas the field in the metallic backed coil is strongly focused to the front of the coil and does not penetrate into the anterior of the coil. As a result, the lack of magnetic field in the anterior of the metallic-backed coil can be used to place components (cables, preamplifiers, etc.) which can be influenced by an external magnetic field, since the interior is shielded from these fields.

The metallic-backed coil shown in FIG. 3B is not completely optimized in performance, because the dielectric layer is too thin, so that currents on the metal surfaces are relatively strong.

FIGS. 3A and 3B illustrate image views of a simulated magnetic field (in Ampere/meter) for (A) non-metal-backed coil (no 30-micrometer copper layer) and (B) metal-backed coil (there is a 30 micrometer copper layer). All other geometric parameters are equivalent. The dielectric region thickness (radius) is 5 mm throughout (from distal to proximal sides of) the coil. Note lack of field protrusion into the coil interior in FIG. 3B. The coil on the right has a gap in imaging intensity in the center of its forward-looking side, and a smaller than optimal forward-looking profile. This is a result of insufficient thickness of the dielectric layer, which can be improved by changing the dielectric region's geometric shape.

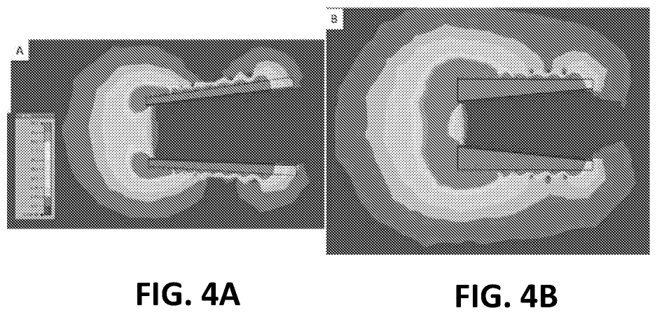

On the Left (Forward) side of the coil of FIG. 3B, because a conous structure is used, the thickness of the dielectric (the distance between the metal surface and the coil windings) can be increased while keeping the coil to a 20 mm outer diameter. FIGS. 4A (same as FIG. 3B) and 4B show the magnetic field effects of increasing the dielectric region's thickness, with FIG. 4B showing a thicker dielectric region on the left (Forward) side, thus increasing the distance between the coil windings and the metallic cone. FIG. 4A illustrates a 5 mm dielectric thickness throughout. FIG. 4B illustrates a 2.5 mm dielectric radius (or thickness) at the proximal (Backward) side of coil, extended to 5 mm radius (or thickness) on the distal (Forward facing) side of the coil. Note the differences in the RF magnetic field extension.

Note that in FIG. 4B the field penetrates further in the forward direction, and the pattern is also more uniform, with no gap in the field intensity in the center of the coil (which can be seen in the coil in FIG. 4A). FIGS. 4A and 4B illustrate image views of magnetic field simulated models for two metal-backed geometries.

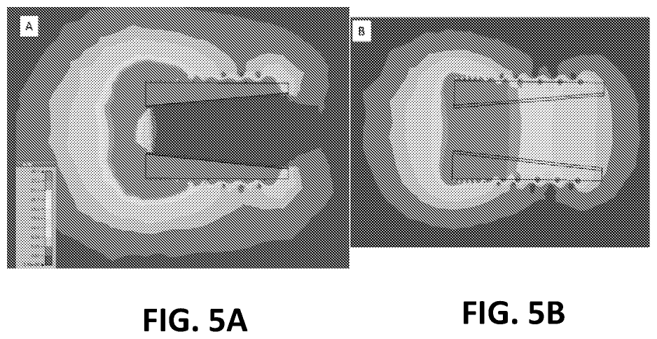

When the issue of dielectric thickness is properly addressed, the metal-backed coil has a far better profile than an equivalent non-metal-backed coil. This can be seen in FIGS. 5A and 5B, where the thickness of the dielectric is now optimal. FIGS. 5A and 5B illustrate image views of RF magnetic-field simulated models for metal-backed versus non-metal backed geometries for the optimally designed coil, which has a 5 mm radius opening on the Forward side, and the thickness of the dialectic varies from 5 mm on the Forward side to 2.5 mm on the Backward side. FIG. 5A illustrates the metal backed geometry, and FIG. 5B illustrates the non-metal backed geometry. Note large differences in RF magnetic field extension. The field lines have been extended forwards by approximately 50%, when comparing the metal-backed versus the non-metal-backed coil.

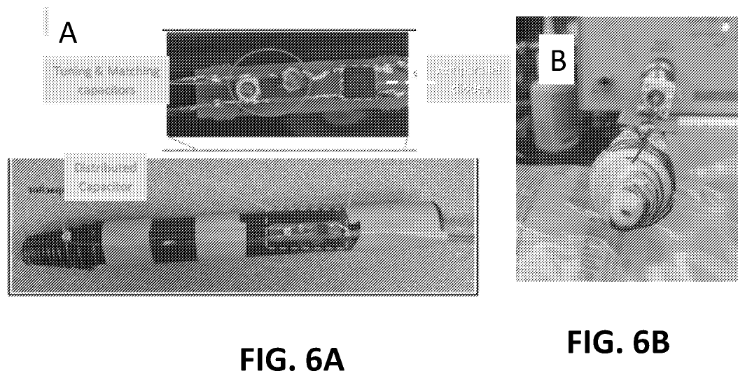

After simulating the designs, varying prototypes of the forward-looking coil are constructed. FIG. 6 shows a typical coil in side and front views. The coils were then tuned and matched to 50 Ohms at 1.5 Tesla (63.8 MHz), for use in a commercial 1.5 Tesla MRI scanner.

FIG. 6A illustrates a side view and FIG. 6B illustrates front views of a 1.5 Tesla coil prototype without the external water-proofing cover layer (which is required to protect the coil's electrical components from fluid present in human tissue). FIG. 6A also shows a circuit board that includes capacitors for tuning the coil to the proper frequency and matching it to an impedance of 50 Ohms, as well as anti-parallel diodes, which are used for passive decoupling of the coil during RF transmission. An additional capacitor is placed in the middle of the coil, in order to distribute the coil's capacitance, thus reducing the RF wave's phase dispersion in the coil and thus improving its operational coherence.

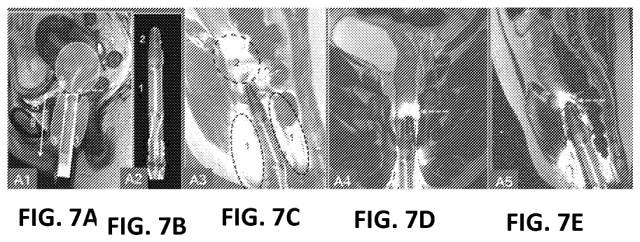

The coils were placed in water phantoms in a 1.5 Tesla scanner, and MRI images were produced, in order to demonstrate the actual lobe pattern of the coils. FIG. 7A shows the intended imaging region of the active obturator coil, which is composed of a sideways-looking array and a forward-looking coil, when placed within the vaginal cavity. The Red dotted region highlights the endometrium, the Yellow arrow highlights the cervix, and the dotted white elliptical regions, numbered 2 and 1, show the lower endometrium and the vaginal wall region, which are the desired regions for imaging. FIG. 7B shows a side view of the active obturator, demonstrating the array elements intended for imaging the posterior endometrium (labeled 2), and those for imaging the vaginal wall, labeled 1. FIG. 7C-7E show the actual imaged region of the array in two large, sexually mature swine.

FIGS. 7A-7E illustrate side views of requirements and design of active obturator. FIG. 7A illustrates that the active obturator is an MRI probe that will be inserted into the vagina and advanced until it is just below the cervix. The coil array includes two separate elements; (1) a sideways-looking coil array to image the walls of the vagina and (2) a forward-looking coil to image the cervix and endometrium. FIGS. 7C-7E illustrate swine imaging results from the array. Note that it effectively images the vaginal walls, and has approximately 4 cm forward-looking ability

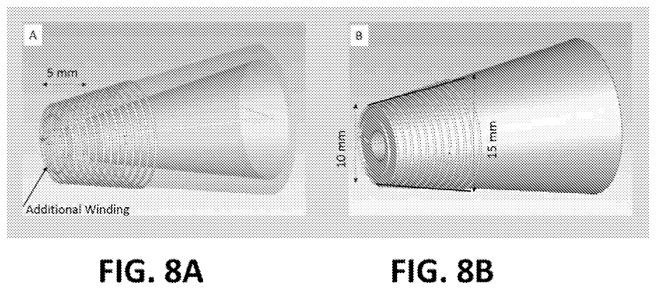

An additional variation on the design of the metal-backed RF coil may include construction of a coil that is dedicated to on-axis visualization. An example of utilization of such a coil could be for placement on the tip of a catheter, with the clinical application being visualization of an occlusion in the blood vessel when the catheter is still a few cm from the location of the occlusion. Such a coil is shown in FIGS. 8A and 8B, where the metallic-layer insert is pulled back from the distal end, ending 5 mm away from the distal end of the coil, a coil winding is added on the distal face of the coil, and the other coil windings are pulled in closer to the metallic layer, which is achieved by reducing the thickness of the dielectric layer.

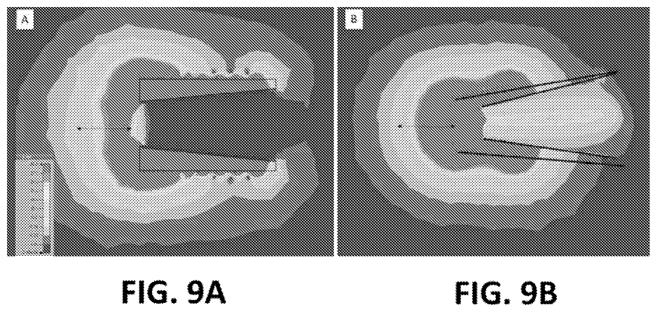

A comparison of the magnetic field profile achieved with the metallic-back coil versus the pulled-back metallic-backed coil is shown in FIGS. 9A and 9B. It can be seen, from the dotted-line with arrows on both ends, that the pulled-back metallic-backed coil looks only forward, and has a greatly more restricted magnetic field towards the sides.

Several electromagnetic simulations were performed. Several prototypes intended for use in 1.5 Tesla Siemens scanner were also constructed, varying several geometric parameters, and the imaging performance of these models recorded. These were used to improve the design further. Non-metal backed prototypes were also constructed (as controls), whose performance was found to be inferior to the preferred design criteria.

Prototypes for 1.5 Tesla Siemens MRI (Aera, Avanto) scanners were constructed, including all of the peripherals required for effective and MRI-safe imaging. The coils included only non-magnetic components, they were tuned and matched for optimal performance, they were decoupled in order to prevent heating during high Specific Absorption Rate (SAR) MRI sequences, and they were connected to a home-built 8-channel MRI receiver.

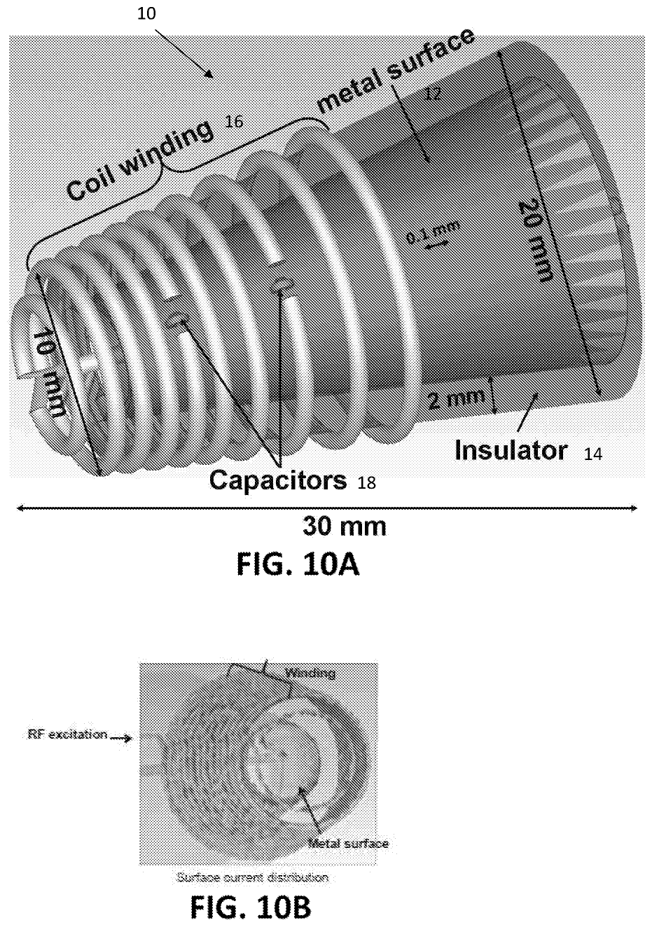

FIG. 10A illustrates a partially sectional view of a forward looking coil design, according to an embodiment of the present invention. The forward looking coil 10 of FIG. 10A includes a metallic surface 12 covered with an insulator 14. A coil winding 16 is disposed on top of the insulator 14. Capacitors 18 are included in the coil windings. The metallic surface 12 creates oppositing time-varying magnetic fields. Properly placed metals can force magnetic fields to project along the desired directions. The coil winding density is higher in the forward direction that in the backward direction. This configuration concentrates the field forwards. The forward-looking coil 10 has a generally conical shape.

FIG. 10B illustrates an image view of surface current distribution on the solenoidal windings and on the metal surface for a forward looking coil design, according to an embodiment of the present invention. The strongest surface currents are colored in red. When an RF current is driven through the windings of a solenoid a primary magnetic field is created. When a metallic cone is placed inside solenoid, the fields induce a surface current on the metal, creating an image magnetic field that exactly cancels the primary field on the metal surface. The primary magnetic field and the image magnetic field are oriented in the same direction outside the solenoid loops, and therefore reinforce the primary field, creating a larger net field.

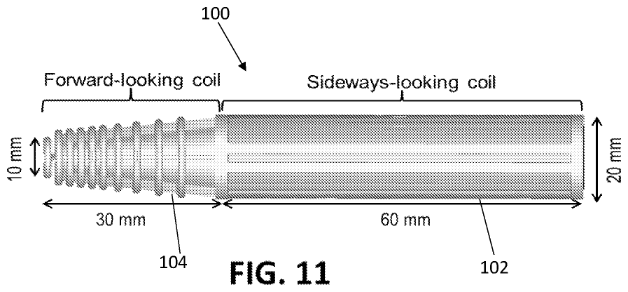

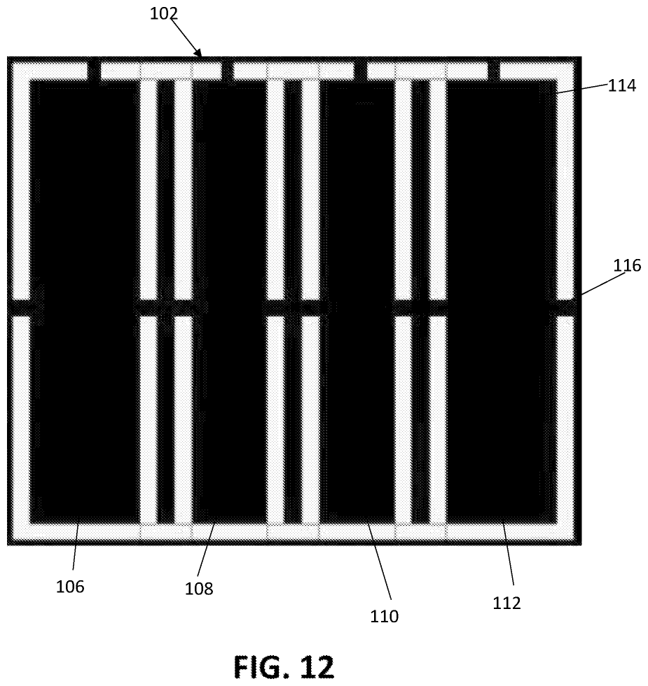

FIG. 11 illustrates a side view of an RF probe, according to an embodiment of the present invention. The entire RF probe 100 includes two arrays. One array is a sideways-looking array 102. This sideways-looking array 102 is configured to image side walls of the anatomy, such as the side walls of the vagina. The sideways-looking array 102 has a generally cylindrical shape. The other array is the forward-looking coil 104 described with respect to FIG. 10A. The sideways-looking array 102 is further described with respect to FIG. 12.

FIG. 12 illustrates a schematic diagram of four elements of a sideways-looking phased array, according to an embodiment of the present invention. The sideways-looking array 102 includes a four-element coil array 106, 108, 110, 112. The four-element coil array 106, 108, 110, 112, is wrapped around a plastic cylinder 114. In some embodiments the plastic cylinder 114 can be 14 cm in length and 2 cm in diameter. The sideways-looking coil array also includes capacitors 116 positioned in the coil windings.

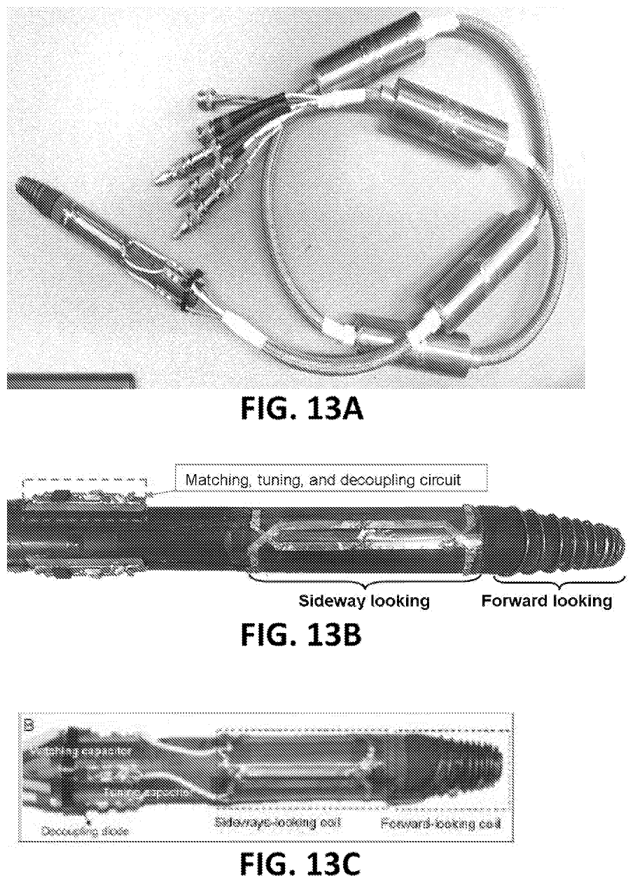

FIGS. 13A-13C illustrate side views of a prototype of an endo-vaginal array, according to an embodiment of the present invention. The “pencil” shaped endo-vaginal array has a cone at its top, for the forward-looking coil, and a cylindrical shaft, for the four-channel sideways-looking array. Its inner open lumen supports its obturator role. The forward-looking coil was designed utilizing the image-magnetic-field concept, wherein properly-positioned metallic surfaces force magnetic fields to project along selected directions. Design specifics were simulated and tested, since closely-placed metals can reduce the coil quality-factor (Q). Finite-element electromagnetic simulations of the forward-looking coil (CST, Germany) evaluated the effect of the metallic surface on the magnetic-field surrounding the coil. The metallic surface shape was simulated, as well as the distance between the metal and the solenoidal coil windings, and the winding diameter and spacing. Optimal designs were then constructed and tested. For the forward-looking coil design: two concentric cone-shaped formers include an inner lumen. The inner former is a 0.5-mm thick metallic cone and the outer former a 2-mm thick plastic cone, with a 10-winding solenoid, with increasing pitch from front to back, wound on its outside. RF phase coherence was maintained with two series caps placed along the coil. For the sideways array design a four-element phased-array sideways-looking coil was added for vaginal wall imaging. The coils, 140-mm long and 20-mm outer diameter, were wrapped at 90-degree increments around the shaft of the device. The device also includes a matching, tuning, and decoupling circuit. The device can be coupled to a braided catheter featuring floating resonant radio-frequency traps (Baluns).

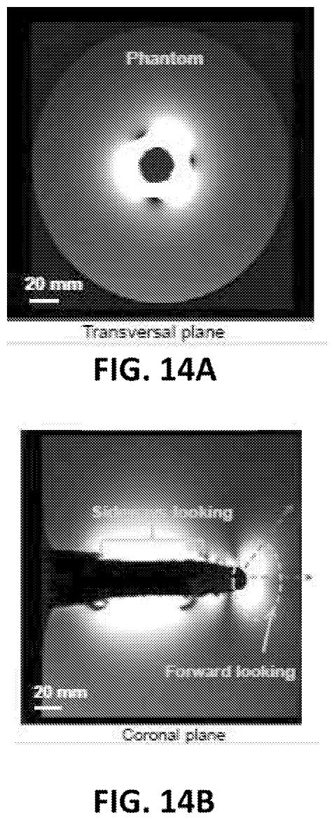

FIGS. 14A and 14B illustrate image views of phantom experiments with a prototype according to an embodiment of the present invention. The phantom experiments validate the simulated magnetic-field profile. Phantom experiments were performed in a 1.5T Siemens MR scanner with the array immersed in a CuSO₄ solution, which mimics the MRI properties of the genitourinary environment. The endo-vaginal coil SNR was calculated, relative to the scanner's 8-channel spine array. A swine experiment was performed to evaluate in-vivo performance. The coil was inserted into the vagina up to the cervix. High-resolution 2D and 3D Fast-Spin-Echo images (TR/TE/=2000 ms/99 ms, slice thickness=3 mm, resolution=0.47×0.47×3.00 mm) were acquired in the coronal plane.

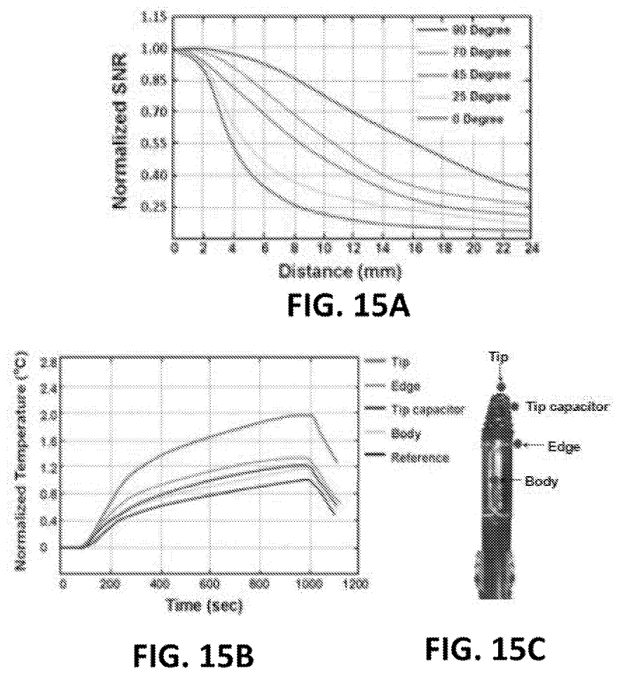

FIG. 15A illustrates a graphical view of measured SNR values along the forward direction, according to an embodiment of the present invention. Measured SNR values along the forward direction of the endo-vaginal coil were 4-8 times higher, relative to the Siemens spine surface coil, over a 20-30 mm region. FIG. 15B illustrates a graphical view of temperature test results, according to an embodiment of the present invention, and FIG. 15C illustrates an endovaginal coil that yielded the results of FIG. 15B. FIG. 15B shows temperature test results during 15 min of a high SAR (3.99 W/Kg) SSFP imaging sequence in an ASTM gel phantom.

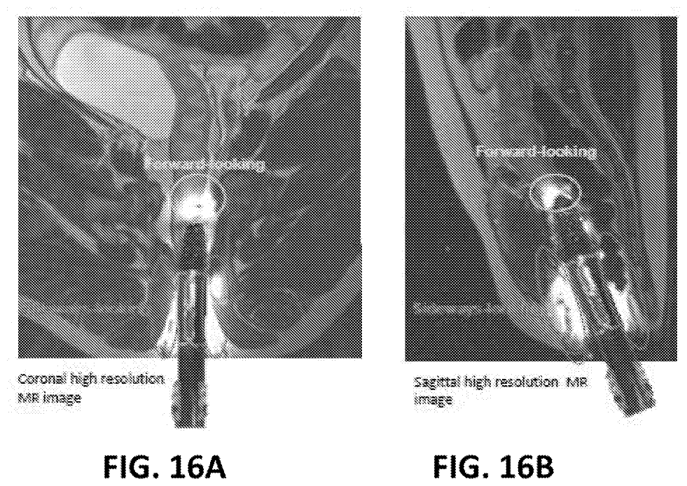

FIGS. 16A-16C illustrate image views from a swine model using the endovaginal coil, according to an embodiment of the present invention. In-vivo swine images acquired with the Endo-vaginal coil demonstrate strong signal hyper-intensity in the vaginal wall and above the vaginal canal.

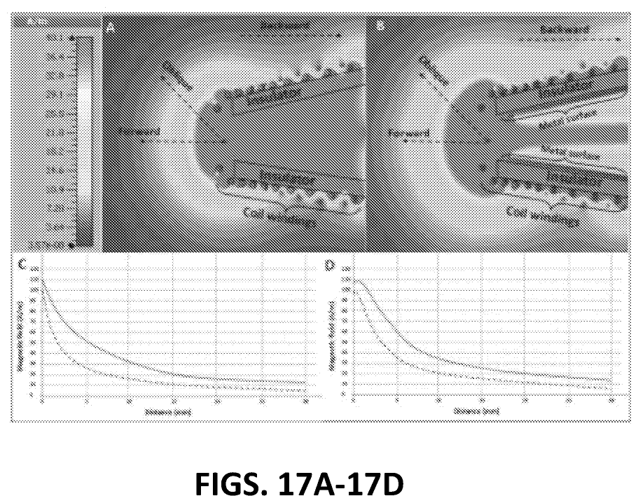

FIGS. 17A-17D illustrate image and graphical views of a comparison of the simulated magnetic field for a non-metallic backed coil, FIG. 17A with a metallic-backed coil, FIG. 17B. Black doted arrows define two directions, the forward and oblique direction. In FIG. 17C the solid line and dashed line show the magnetic field profiles of the metallic-backed coil and non-metallic backed coil, respectively, in the forward direction. FIG. 17D illustrates the solid line and dashed line that show the magnetic field profiles of the metallic-backed coil and nonmetallic backed coil, respectively, in the oblique direction. FIGS. 17A-17D show simulated magnetic-field profiles for a metal-backed versus a non-metal backed construct. The field lines in the forward and oblique directions extended further forwards in the metal-backed versus the non-metal-backed cases.

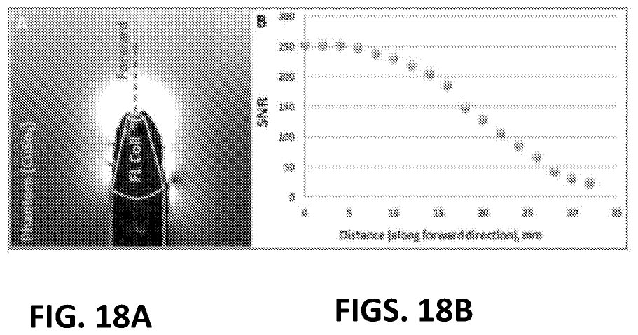

FIG. 18A illustrates a 2D FSE image of the Forward-looking coil in a (copper sulfate) CuSO₄-solution phantom combining the forward-looking looking coil and the scanner's spine-array coils. FIG. 18B illustrates the calculated SNR profile in the forward direction, which shows 4-8 times the spine-coil SNR enhancement. The endo-vaginal array of the present invention therefore includes a forward-looking coil that provides 4-8 times surface-coil SNR at distances of 20-30 mm above the coil. The array can be used during planning as well as during radiation delivery.

The control of the present invention can be carried out using a computer, non-transitory computer readable medium, or alternately a computing device or non-transitory computer readable medium incorporated into the robotic device. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. The operating console for the device is a non-generic computer specifically designed by the manufacturer. It is not a standard business or personal computer that can be purchased at a local store. Additionally, the console computer can carry out communications through the execution of proprietary custom built software that is designed and written by the manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device for magnetic resonance imaging comprising:
an extended, forward-looking radio-frequency (RF) coil for use in imaging and visualization;
a base for the extended, forward-looking RF coil; and
a metallic layer deposited on the base, such that it provides a metallic backing for the extended, forward-looking RF coil.

2. The device of claim 1 further comprising the extended, forward looking RF coil including an inner void.

3. The device of claim 2 wherein the extended, forward looking RF coil is surrounded by the base taking a form of a cone-shaped plastic enclosure.

4. The device of claim 3 wherein the metallic layer is deposited on an outer surface of the cone.

5. The device of claim 1 further comprising an obturator.

6. The device of claim 1 further comprising coil windings of the extended, forward-looking RF coil being denser on a left side (forward side) as compared to the right side (backwards side).

7. The device of claim 6 further comprising capacitors disposed in the coil windings.

8. The device of claim 1 further comprising a matching, tuning, and decoupling circuit.

9. The device of claim 1 further comprising a braided metallic catheter including floating resonant radio-frequency traps (Baluns).

10. A device for magnetic resonance imaging comprising:
a forward-looking magnetic resonance imaging array, wherein the forward-looking magnetic resonance imaging array includes a metallic layer and coil windings disposed outside of the metallic layer;
a sideways-looking magnetic resonance imaging array, wherein the sideways-looking magnetic resonance imaging array includes electrically-conductive coils disposed about a base.

11. The device of claim 10 further comprising the coil windings of the forward-looking magnetic resonance imaging array being more dense at a distal end of the array that at a proximal end of the array.

12. The device of claim 10 further comprising capacitors disposed within the coil windings of the forward-looking magnetic resonance imaging array.

13. The device of claim 10 further comprising an insulator layer disposed between the metallic layer and the coil windings of the forward-looking magnetic resonance imaging array.

14. The device of claim 10 wherein the forward-looking magnetic resonance imaging array takes a generally conical form.

14. The device of claim 10 wherein the coil windings of the sideways-looking array are disposed around a plastic cylinder.

15. The device of claim 10 wherein the coil windings of the sideways-looking array includes capacitors disposed among the coil windings.

16. The device of claim 10 wherein the sideways-looking magnetic resonance imaging array includes a four-element coil array.

17. The device of claim 16 wherein each element of the four-element coil array covers a 90 degree arc.

18. The device of claim 10 further comprising a matching, tuning, and decoupling circuit.

19. The device of claim 10 further comprising a metallic-braided cable assembly including floating resonant radio-frequency traps (Baluns) mounted on its shaft.

20. The device of claim 10 wherein the forward-looking magnetic resonance imaging array and the sideways-looking magnetic resonance imaging array are configured for imaging the vagina and cervix.
